(19) United States
(12) Patent Application Publication
(10) Pub. No.: US 2022/0071887 A1
(43) Pub. Date: Mar. 10, 2022

(54) NAIL COATING REMOVER CREAM MASK AND THE METHOD OF USING THEREOF

(71) Applicant: LiJuan Zhen, Brea, CA (US)

(72) Inventor: LiJuan Zhen, Brea, CA (US)

(21) Appl. No.: 17/013,541

(22) Filed: Sep. 5, 2020

(51) Int. Cl. A61K 8/87; A61K 8/67; A61K 8/81; A61K 8/35; A61K 8/37; A61K 8/88; A61K 8/34; A61Q 3/04; A61K 8/44; A61K 8/85

(52) U.S. Cl. CPC A61K 8/87; A61K 8/671; A61K 8/8194; A61K 8/35; A61K 8/37; A61K 8/88; A61K 2800/59; A61K 8/676; A61Q 3/04; A61K 8/44; A61K 8/85; A61K 8/8152; A61K 8/345

(57) ABSTRACT

A nail coating remover cream mask comprises a film forming agent from about 5% to about 70% by weight, relative to the total weight of the nail coating remover cream mask, water from about 2% to about 45% by weight, relative to the total weight of the nail coating remover cream mask, a first solvent from about 2% to about 45% by weight, relative to the total weight of the nail coating remover cream mask, a second solvent from about 2% to about 80% by weight, relative to the total weight of the nail coating remover cream mask, a third solvent from about 2% to about 80% by weight, relative to the total weight of the nail coating remover cream mask, and a moisture agent from about 0.1% to about 5% by weight, relative to the total weight of the nail coating remover cream mask, and a method to use the nail coating remover cream mask to remove nail coating.

### CROSS-REFERENCE RELATED TO RELATED APPLICATIONS

None.

### BACKGROUND

Nail coating, which includes all types of nail coating, such as but not limited to nail polish, nail gel, nail dip powder, may not be easily removed and can be a topcoat, color coat, or base coat. A traditionally commonly used nail coating remover is acetone. However, acetone needs to be soaked in a cotton ball or a cotton pad or similar materials. The cotton ball needs to be placed on the nail coat which is on the nail. After placing the cotton ball soaked with acetone on the nail coating, the fingertip of the nail needs to be wrapped by aluminum foil for a waiting duration. After the waiting duration, a tool is required to scrape off the loosen nail coating, which will greatly damage the nail. Also, if the nail coating is a topcoat on a color coat instead of merely a color coat, the topcoat needs to be buffed off by a buffer before the application of acetone on the color coat.

### BRIEF SUMMARY OF THE INVENTION

The invention is directed to nail coating remover cream mask comprises a film forming agent from about 5% to about 70% by weight, relative to the total weight of the nail coating remover cream mask, water from about 2% to about 45% by weight, relative to the total weight of the nail coating remover cream mask, a first solvent from about 2% to about 45% by weight, relative to the total weight of the nail coating remover cream mask, a second solvent from about 2% to about 80% by weight, relative to the total weight of the nail coating remover cream mask, a third solvent from about 2% to about 80% by weight, relative to the total weight of the nail coating remover cream mask, and a moisture agent from about 0.1% to about 5% by weight, relative to the total weight of the nail coating remover cream mask. The invention is also directed to a method to remove a nail coating from a nail comprises (a) applying a nail coating remover cream mask on the nail coating, wherein the nail coating remover cream mask comprising a film forming agent from about 5% to about 70% by weight, relative to the total weight of the nail coating remover cream mask, water from about 2% to about 45% by weight, relative to the total weight of the nail coating remover cream mask, a first solvent from about 2% to about 45% by weight, relative to the total weight of the nail coating remover cream mask, a second solvent from about 2% to about 80% by weight, relative to the total weight of the nail coating remover cream mask, a third solvent from about 2% to about 80% by weight, relative to the total weight of the nail coating remover cream mask, and a moisture agent from about 0.1% to about 5% by weight, relative to the total weight of the nail coating remover cream mask; (b) forming a film on the nail coating remover cream mask when the nail coating remover cream mask is applied on a nail coating, wherein the film is formed in approximately 30 seconds to 2 minutes, wherein the film will contain the nail coating remover cream mask in a liquid state lasting for approximately 45 minutes to 60 minutes above the nail coating; (c) remaining the nail coating remover cream mask on the nail coating for approximately 5 minutes to 10 minutes, wherein the nail coating remover cream mask reacts with the nail coating on the nail, wherein the nail coating will be dissolved by the nail coating remover cream mask in the liquid state; and (d) removing the nail coating remover cream mask and the nail coating that has been dissolved.

It is to be understood that both the foregoing general description and the following detailed description are exemplary and explanatory only and are not restrictive of the invention.

### DETAILED DESCRIPTION

Before the present invention is described in greater detail, it is to be understood that this invention is not limited to particular embodiments described, and as such may, of course, vary. It is also to be understood that the terminology used herein is for the purpose of describing particular embodiments only, and is not intended to be limiting, since the scope of the present invention will be limited only by the appended claims.

Wherein a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” may also include the plural referents unless the context clearly dictates otherwise.

It is further noted that the claims may be drafted to exclude any element that may be optional. As such, this statement is intended to serve as antecedent basis for use of such exclusive terminology as “solely”, “only” and the like in connection with the recitation of claim elements, or the use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

The disclosure is related a nail coating remover cream mask. In one embodiment, the nail coating remover cream mask comprises nail coating remover cream mask comprises a film forming agent from about 5% to about 70% by weight, relative to the total weight of the nail coating remover cream mask, water from about 2% to about 45% by weight, relative to the total weight of the nail coating remover cream mask, a first solvent from about 2% to about 45% by weight, relative to the total weight of the nail coating remover cream mask, a second solvent from about 2% to about 80% by weight, relative to the total weight of the nail coating remover cream mask, a third solvent from about 2% to about 80% by weight, relative to the total weight of the nail coating remover cream mask, and a moisture agent from about 0.1% to about 5% by weight, relative to the total weight of the nail coating remover cream mask. The nail coating remover cream mask is in a form of a cream that can be a liquid state but can form a film on the top of the cream to contain the rest of the cream in a liquid state under the film.

In one embodiment, the film forming agent is selected from the group consisting of polyurethane polymer, olyureas, polyesters, polyesteramides, acrylic polymer, aliphatic urethane acrylate, propoxylated neopentyle glycol diacrylate, trimethylbenzoyl diphenylphosphine oxide, methyl methacrylate, ethyl methacrylate, butyl methacrylate, isobutyl methacrylate, 2-ethylhexylmeth acrylate and lauryl methacrylate and their mixtures thereof. In one preferred embodiment of the present invention, the film forming agent is polyurethane. In another preferred embodiment of the present invention, the film forming agent is acrylic polymer. The film forming agent will be under a polymerization to form a film on the surface of the nail coating remover cream mask when the nail coating remover cream mask is applied on a nail coating.

In one embodiment of the present invention, the first solvent is selected from the group consisting of ethyl alcohol, isopropanol, diacetone alcohol, 2-butoxyethanol, cyclohexanol and their mixtures thereof. The first solvent can be miscible in oil or water. In one preferred embodiment of the present invention, the first solvent is ethyl alcohol. Ethyl alcohol contains a polar and a nonpolar group. Therefore, ethyl alcohol can dissolve polar or nonpolar chemical compounds. Ethyl alcohol can be a solvent or carrier for the mixture of polymer into a solution mixture, like the nail coating remover cream mask.

In one embodiment of the present invention, the second solvent is selected from the group consisting of ethyl acetate, methyl acetate, propyl acetate, isopropyl acetate, n-butyl acetate, iso-pentyl acetate, methoxypropyl acetate, t-butyl acetate, butyl lactate, isoprene glycol, butylene glycol, propylene glycol, glycerol, sorbitol, polyethylene glycols, dipropylene glycol methyl ether, dipropylene glycol monobutyl ether and their mixtures thereof. The second solvent can be miscible in oil or water. In one preferred embodiment of the present invention, the second solvent is ethyl acetate. Ethyl acetate is used as one type of nail coating removal chemicals.

In one embodiment of the present invention, the third solvent is selected from acetone, ester, ethyl ketone, methyl isobutyl ketone, methyl ethyl ketone, diisobutyl ketone, isophorone, cyclohexanone, dihydroxyacetone, ethyl methyl valerolactone, ethyl butyl valerolactone and their mixtures thereof. The third solvent can be miscible in oil or water. In one preferred embodiment of the present invention, the third solvent is acetone, which is a traditional nail coating remover. It is essential that solvents is kept in the cream of the nail coating remover cream mask so that they can react with the nail coatings, regardless the nail coating is formed by nail polish, nail gel, nail dip powder, or nail spray, and regardless the nail coating is a topcoat, a color coat, or a base coat. The color coat can be directed on the nail surface, but some types of the nail polish, nail gel, nail dip powder or nail spray will prefer a base coat to be applied on the nail surface, a color coat to be applied on the base coat, and a topcoat to be applied on the color coat to last the life and endurance of the color coats on the nail.

In one embodiment of the present invention, the moisture agent is selected from the group consisting of hyaluronic acid, amino acids, aminoacetic acid, vitamin A, vitamin C, vitamin D, d-alpha-tocopherol, magnesium 1-ascorbyl-2-phosphate, ferulic acid, ascorbic acid, ubiquinone, allantoin, 2,5-dioxo-4-imidazolidinyl, 2,3-dihydroxypropyl ester octadecanoic acid, superoxide dismutase, tocopheryl acetate, squalane (tetracosane), urea perhydrate, bioflavonoids, acetyl tyrosine, lecithin, polymethylsiloxane, glycerol (1,2,3-trihydroxypropane), extract of fruits, extract of plants and their mixtures thereof. In one preferred embodiment, the moisture agent is hyaluronic acid, which locks moisture on the nail surface because solvents will dry the nail surface when they react with acetone.

In one embodiment, the nail coating remover cream mask may optionally comprise fragrance. The fragrance is a common additive to cosmetic products. A fragrance can be synthetic or natural extract. A person having ordinary skill in the art will understand the fragrance that can be used in cosmetic products.

In one embodiment, a film is formed on the nail coating remover cream mask when the nail coating remover cream mask is applied on a nail coating, wherein the film is formed in approximately 30 seconds to 2 minutes, wherein the film will contain the nail coating remover cream mask in a liquid state lasting for approximately 45 minutes to 60 minutes above the nail coating, wherein the nail coating remover cream mask reacts with the nail coating on the nail, wherein the nail coating will be dissolved by the nail coating remover cream mask in the liquid state, and wherein the nail coating remover cream mask and the nail coating that has been dissolved can be removed from the nail between approximately 5 minutes to 10 minutes. The film by the film forming polymer that is formed on top of the nail coating remover cream mask will prevent the solvents in the nail coating remover cream mask from being volatized from the nail coating remover cream mask. To keep the nail coating remover cream mask is a liquid state that contains solvent is essential because the solvents are needed to dissolve the nail coating. The film also acts a wrap to prevent from smearing other objects when the nail coating remover cream mask is touched by other objects such as fingers, skins, or cloths.

The current invention is also directed to a method to remove a nail coating from a nail. In one embodiment of the current invention, the method comprises step (a) that a nail coating remover cream mask is applied on the nail coating, wherein the nail coating remover cream mask comprising a film forming agent from about 5% to about 70% by weight, relative to the total weight of the nail coating remover cream mask, water from about 2% to about 45% by weight, relative to the total weight of the nail coating remover cream mask, a first solvent from about 2% to about 45% by weight, relative to the total weight of the nail coating remover cream mask, a second solvent from about 2% to about 80% by weight, relative to the total weight of the nail coating remover cream mask, a third solvent from about 2% to about 80% by weight, relative to the total weight of the nail coating remover cream mask, and a moisture agent from about 0.1% to about 5% by weight, relative to the total weight of the nail coating remover cream mask; a step (b) to form a film on the nail coating remover cream mask when the nail coating remover cream mask is applied on a nail coating, wherein the film is formed in approximately 30 seconds to 2 minutes, wherein the film will contain the nail coating remover cream mask in a liquid state lasting for approximately 45 minutes to 60 minutes above the nail coating; a step (c) to remain the nail coating remover cream mask on the nail coating for approximately 5 minutes to 10 minutes, wherein the nail coating remover cream mask reacts with the nail coating on the nail, wherein the nail coating will be dissolved by the nail coating remover cream mask in the liquid state; and a step (d) that the nail coating remover cream mask is removed with the nail coating that has been dissolved.

In one embodiment of the invention, the film forming agent is selected from the group consisting of polyurethane polymer, olyureas, polyesters, polyesteramides, acrylic polymer, aliphatic urethane acrylate, propoxylated neopentyle glycol diacrylate, trimethylbenzoyl diphenylphosphine oxide, methyl methacrylate, ethyl methacrylate, butyl methacrylate, isobutyl methacrylate, 2-ethylhexylmeth acrylate and lauryl methacrylate and their mixtures thereof. In one embodiment, the first solvent is selected from the group consisting of ethyl alcohol, isopropanol, diacetone alcohol, 2-butoxyethanol, cyclohexanol and their mixtures thereof. In one embodiment, the second solvent is selected from the group consisting of ethyl acetate, ethyl acetate, methyl acetate, propyl acetate, isopropyl acetate, n-butyl acetate, iso-pentyl acetate, methoxypropyl acetate, t-butyl acetate, butyl lactate, isoprene glycol, butylene glycol, propylene glycol, glycerol, sorbitol, polyethylene glycols, dipropylene glycol methyl ether, dipropylene glycol monobutyl ether and their mixtures thereof. In one embodiment, the third solvent is selected from acetone, ester, ethyl ketone, methyl isobutyl ketone, methyl ethyl ketone, diisobutyl ketone, isophorone, cyclohexanone, dihydroxyacetone, ethyl methyl valerolactone, ethyl butyl valerolactone and their mixtures thereof. In one embodiment, the moisture agent is selected from the group consisting of hyaluronic acid, amino acids, aminoacetic acid, vitamin A, vitamin C, vitamin D, d-alpha-tocopherol, magnesium l-ascorbyl-2-phosphate, ferulic acid, ascorbic acid, ubiquinone, allantoin, 2,5-dioxo-4-imidazolidinyl, 2,3-dihydroxypropyl ester octadecanoic acid, superoxide dismutase, tocopheryl acetate, squalane (tetracosane), urea perhydrate, bioflavonoids, acetyl tyrosine, lecithin, polymethylsiloxane, glycerol (1,2,3-trihydroxypropane), extract of fruits, extract of plants and their mixtures thereof.

In one preferred embodiment, the film forming agent, the first solvent, the second solvent, and the third solvent are polyurethane, ethyl alcohol, ethyl acetate, and acetone, respectively. In one preferred embodiment, the moisture agent is hyaluronic acid. In one embodiment, the nail coating remover cream mask may optionally comprise fragrance. The fragrance is a common additive to cosmetic products. A fragrance can be synthetic or natural extract. A person having ordinary skill in the art will understand the fragrance that can be used in cosmetic products.

In one embodiment, the method to remove a nail coating from a nail of claim 9, further comprising a step of buffing off a topcoat before the step to apply the nail coating remover cream mask on the nail coating. The topcoat is optionally to be removed. If the topcoat is not removed, the time to remain the nail coating remover cream mask on the nail coating that has the topcoat will be longer, such as 10 minutes before the nail coating remover cream mask to be removed from the nail.

The maximum time to form the film and the maximum time to remove the coating remover cream mask that has reacts with the nail coating from the nail depends on the concentrations of the film forming agent, water, and the first, second, and third solvents. In one preferred embodiment, the film can be formed between 30 seconds and 1 minute and the nail coating remover cream mask that reacts with the nail coat can be removed from nail when the film forming agent being more in the nail coating remover cream mask, such as 60% to 70% by weight, relative to the total weight of the nail coating remover cream mask and the second and the third solvent are at 2% to 80%, relative to the total weight of the nail coating remover cream mask. A table of examples of embodiments showing the ranges of the ingredients versus the maximum time to form the film and the maximum time to remove the nail coat is as the following TABLE 1. In TABLE 1, “Min.” means minutes. “Maximum time to remove” means the required time before the nail coating remover cream mask can be removed from the nail. “INCI Name” means the name of the chemicals following the convention by the International Nomenclature Cosmetic Ingredient.

TABLE 1

| | Example 1 | Example 2 | Example 3 |
|---|---|---|---|
| INCI Name | % by Weight | % by Weight | % by Weight |
| film forming agent | 5%-50% | 5%-60% | 5%-70% |
| water | 2%-25% | 2%-35% | 2%-45% |
| first solvent | 2%-25% | 2%-35% | 2%-45% |
| second solvent | 2%-50% | 2%-60% | 2%-80% |
| second solvent | 2%-50% | 2%-60% | 2%-80% |
| Moisture agent | 0.1%-5% | 0.1%-5% | 0.1%-5% |
| maximum time to form the | 2 min. | 1.5 min. | 1 min. |
| film | | | |
| maximum time to remove | 10 min. | 8 min. | 5 min. |

As stated above, the embodiment of the method to remove nail coat will be different from the traditional way of applying acetone that needs wrapping of aluminum around the finger tip to keep the acetone that is soaked in a cotton ball or pad from being volatized and keep the acetone continuously contact with the nail coat to react.

1. A nail coating remover cream mask, comprising:
a film forming agent from about 5% to about 70% by weight, relative to the total weight of the nail coating remover cream mask,
water from about 2% to about 45% by weight, relative to the total weight of the nail coating remover cream mask,
a first solvent from about 2% to about 45% by weight, relative to the total weight of the nail coating remover cream mask,
a second solvent from about 2% to about 80% by weight, relative to the total weight of the nail coating remover cream mask,
a third solvent from about 2% to about 80% by weight, relative to the total weight of the nail coating remover cream mask, and
a moisture agent from about 0.1% to about 5% by weight, relative to the total weight of the nail coating remover cream mask.

2. The nail coating remover cream mask of claim 1,
wherein the film forming agent is selected from the group consisting of polyurethane polymer, olyureas, polyesters, polyesteramides, acrylic polymer, aliphatic urethane acrylate, propoxylated neopentyl glycol diacrylate, trimethylbenzoyl diphenylphosphine oxide, methyl methacrylate, ethyl methacrylate, butyl methacrylate, isobutyl methacrylate, 2-ethylhexylmeth acrylate and lauryl methacrylate and their mixtures thereof;
wherein the first solvent is selected from the group consisting of ethyl alcohol, isopropanol, diacetone alcohol, 2-butoxyethanol, cyclohexanol and their mixtures thereof;
wherein the second solvent is selected from the group consisting of ethyl acetate, methyl acetate, propyl acetate, isopropyl acetate, n-butyl acetate, iso-pentyl acetate, methoxypropyl acetate, t-butyl acetate, butyl lactate, isoprene glycol, butylene glycol, propylene glycol, glycerol, sorbitol, polyethylene glycols, dipropylene glycol methyl ether, dipropylene glycol monobutyl ether and their mixtures thereof;
wherein the third solvent is selected from acetone, ester, ethyl ketone, methyl isobutyl ketone, methyl ethyl ketone, diisobutyl ketone, isophorone, cyclohexanone, dihydroxyacetone, ethyl methyl valerolactone, ethyl butyl valerolactone and their mixtures thereof; and
wherein the moisture agent is selected from the group consisting of hyaluronic acid, amino acids, aminoacetic acid, vitamin A, vitamin C, vitamin D, d-alpha-tocopherol, magnesium 1-ascorbyl-2-phosphate, ferulic acid, ascorbic acid, ubiquinone, allantoin, 2,5-dioxo-4-imidazolidinyl, 2,3-dihydroxypropyl ester octadecanoic acid, superoxide dismutase, tocopheryl acetate, squalane (tetracosane), urea perhydrate, bioflavonoids, acetyl tyrosine, lecithin, polymethylsiloxane, glycerol (1,2,3-trihydroxypropane), extract of fruits, extract of plants and their mixtures thereof.

3. The nail coating remover cream mask of claim 1, further comprising a fragrance.

4. The nail coating remover cream mask of claim 1,
wherein a film is formed on the nail coating remover cream mask when the nail coating remover cream mask is applied on a nail coating,
wherein the film is formed in approximately 30 seconds to 2 minutes,
wherein the film will contain the nail coating remover cream mask in a liquid state lasting for approximately 45 minutes to 60 minutes above the nail coating,
wherein the nail coating remover cream mask reacts with the nail coating on the nail,
wherein the nail coating will be dissolved by the nail coating remover cream mask in the liquid state, and
wherein the nail coating remover cream mask and the nail coating that has been dissolved can be removed from the nail between approximately 5 minutes to 10 minutes.

5. A nail coating remover cream mask, comprising,
a film forming agent is about 5% to about 70% by weight, relative to the total weight of the nail coating remover cream mask;
water from about 2% to about 45% by weight, relative to the total weight of the nail coating remover cream mask,
a first solvent from about 2% to about 45% by weight, relative to the total weight of the nail coating remover cream mask;
a second solvent from about 2% to about 80% by weight, relative to the total weight of the nail coating remover cream mask;
a third solvent from about 2% to about 80% by weight, relative to the total weight of the nail coating remover cream mask; and
a moisture agent from about 0.1% to about 5% by weight, relative to the total weight of the nail coating remover cream mask.

6. The nail coating remover cream mask of claim 5,
wherein the film forming agent is selected from the group consisting of polyurethane polymer, olyureas, polyesters, polyesteramides, acrylic polymer, aliphatic urethane acrylate, propoxylated neopentyle glycol diacrylate, trimethylbenzoyl diphenylphosphine oxide, methyl methacrylate, ethyl methacrylate, butyl methacrylate, isobutyl methacrylate, 2-ethylhexylmeth acrylate and lauryl methacrylate and their mixtures thereof;
wherein the first solvent is selected from the group consisting of ethyl alcohol, isopropanol, diacetone alcohol, 2-butoxyethanol, cyclohexanol and their mixtures thereof;
wherein the second solvent is selected from the group consisting of ethyl acetate, ethyl acetate, methyl acetate, propyl acetate, isopropyl acetate, n-butyl acetate, iso-pentyl acetate, methoxypropyl acetate, t-butyl acetate, butyl lactate, isoprene glycol, butylene glycol, propylene glycol, glycerol, sorbitol, polyethylene glycols, dipropylene glycol methyl ether, dipropylene glycol monobutyl ether and their mixtures thereof;
wherein the third solvent is selected from acetone, ester, ethyl ketone, methyl isobutyl ketone, methyl ethyl ketone, diisobutyl ketone, isophorone, cyclohexanone, dihydroxyacetone, ethyl methyl valerolactone, ethyl butyl valerolactone and their mixtures thereof; and
wherein the moisture agent is selected from the group consisting of hyaluronic acid, amino acids, aminoacetic acid, vitamin A, vitamin C, vitamin D, d-alpha-tocopherol, magnesium 1-ascorbyl-2-phosphate, ferulic acid, ascorbic acid, ubiquinone, allantoin, 2,5-dioxo-4-imidazolidinyl, 2,3-dihydroxypropyl ester octadecanoic acid, superoxide dismutase, tocopheryl acetate, squalane (tetracosane), urea perhydrate, bioflavonoids, acetyl tyrosine, lecithin, polymethylsiloxane, glycerol (1,2,3-trihydroxypropane), extract of fruits, extract of plants and their mixtures thereof.

7. The nail coating remover cream mask of claim 5, further comprising a fragrance.

8. The nail coating remover cream mask of claim 5,
wherein a film is formed on the nail coating remover cream mask when the nail coating remover cream mask is applied on a nail coating,
wherein the film is formed in approximately 30 seconds to 1 minute,
wherein the film will contain the nail coating remover cream mask in a liquid state lasting for approximately 45 minutes to 60 minutes above the nail coating,
wherein the nail coating remover cream mask reacts with the nail coating on the nail,
wherein the nail coating will be dissolved by the nail coating remover cream mask in the liquid state, and
wherein the nail coating remover cream mask and the nail coating that has been dissolved can be removed from the nail approximately in 5 minutes.

9. A method to remove a nail coating from a nail, comprising:
(a) applying a nail coating remover cream mask on the nail coating, wherein the nail coating remover cream mask comprises a film forming agent from about 40% to about 70% by weight, relative to the total weight of the nail coating remover cream mask, water from about 2% to about 45% by weight, relative to the total weight of the nail coating remover cream mask, a first solvent from about 2% to about 45% by weight, relative to the total weight of the nail coating remover cream mask, a second solvent from about 2% to about 80% by weight, relative to the total weight of the nail coating remover cream mask, a third solvent from about 2% to about 80% by weight, relative to the total weight of the nail coating remover cream mask, and a moisture agent from about 0.1% to about 5% by weight, relative to the total weight of the nail coating remover cream mask;
(b) forming a film on the nail coating remover cream mask when the nail coating remover cream mask is applied on a nail coating, wherein the film is formed in approximately 30 seconds to 2 minutes, wherein the film will contain the nail coating remover cream mask in a liquid state lasting for approximately 45 minutes to 60 minutes above the nail coating;
(c) maintaining the nail coating remover cream mask on the nail coating for approximately 5 minutes to 10 minutes, wherein the nail coating remover cream mask reacts with the nail coating on the nail, wherein the nail coating will be dissolved by the nail coating remover cream mask in the liquid state; and
(d) removing the nail coating remover cream mask with the nail coating that has been dissolved.

10. The method to remove a nail coating from a nail of claim 9,
wherein the film forming agent is selected from the group consisting of polyurethane polymer, polyureas, polyesters, polyesteramides, acrylic polymer, aliphatic urethane acrylate, propoxylated neopentyl glycol diacrylate, trimethylbenzoyl diphenylphosphine oxide, methyl methacrylate, ethyl methacrylate, butyl methacrylate, isobutyl methacrylate, 2-ethylhexylmeth acrylate and lauryl methacrylate and their mixtures thereof;
wherein the first solvent is selected from the group consisting of ethyl alcohol, isopropanol, diacetone alcohol, 2-butoxyethanol, cyclohexanol and their mixtures thereof;
wherein the second solvent is selected from the group consisting of ethyl acetate, methyl acetate, propyl acetate, isopropyl acetate, n-butyl acetate, iso-pentyl acetate, methoxypropyl acetate, t-butyl acetate, butyl lactate, isoprene glycol, butylene glycol, propylene glycol, glycerol, sorbitol, polyethylene glycols, dipropylene glycol methyl ether, dipropylene glycol monobutyl ether and their mixtures thereof;
wherein the third solvent is selected from acetone, ester solvents, ethyl ketone, methyl isobutyl ketone, methyl ethyl ketone, diisobutyl ketone, isophorone, cyclohexanone, dihydroxyacetone, ethyl methyl valerolactone, ethyl butyl valerolactone and their mixtures thereof; and
wherein the moisture agent is selected from the group consisting of hyaluronic acid, amino acids, aminoacetic acid, vitamin A, vitamin C, vitamin D, d-alpha-tocopherol, magnesium 1-ascorbyl-2-phosphate, ferulic acid, ubiquinone, allantoin, 2,5-dioxo-4-imidazolidinyl, 2,3-dihydroxypropyl ester of octadecanoic acid, superoxide dismutase, tocopheryl acetate, squalane, urea perhydrate, bioflavonoids, acetyl tyrosine, lecithin, polymethylsiloxane, glycerol, extract of fruits, extract of plants and their mixtures thereof.

11. The method to remove a nail coating from a nail of claim 9,
wherein the nail coating remover cream mask further comprises a fragrance.

12. The method to remove a nail coating from a nail of claim 9, further comprising a step of buffing off a topcoat before applying the nail coating remover cream mask on the nail coating.
